(19) United States
(12) Patent Application Publication
(10) Pub. No.: US 2003/0224055 A1
(43) Pub. Date: Dec. 4, 2003

(54) Compositions and processes for inhibiting gene expression using polynucleotides

(76) Inventors: David L. Lewis, Madison, WI (US); David B. Rozema, Madison, WI (US); Darren Wakefield, Madison, WI (US); Hans Herweijer, Madison, WI (US); Jon A. Wolff, Madison, WI (US); James E. Hagstrom, Middleton, WI (US)

Correspondence Address: Mark K. Johnson, Mirus Corporation, 505 S. Rosa Rd., Madison, WI, 53719, US

(21) Appl. No.: 10/446,252

(22) Filed: May 28, 2003

Related U.S. Application Data
(60) Provisional application No. 60/383,994, filed on May 28, 2002.

(51) Int. Cl. A61K 38/16; A61K 48/00; A61K 9/14; C12N 15/85

(52) U.S. Cl. 424/486; 435/455; 514/12; 514/81

(57) ABSTRACT

Compositions are provided for delivery of polynucleotides to cells for the purpose of inhibiting gene expression. Antisense polynucleotide-containing complexes are described. The salt and serum stability and small size of the complexes permits delivery to cells in vitro and in vivo.

### CROSS-REFERENCE TO RELATED APPLICATIONS

This application is related to prior provisional application No. 60/383,994 filed May 28, 2002.

### BACKGROUND

Most genes function by expressing a protein via an intermediate, termed messenger RNA (mRNA) or sense RNA. The ability to specifically knock-down expression of a target gene by anti-mRNA agents has obvious benefits. For example, anti-mRNA agents could be used to generate animals that mimic true genetic “knockout” animals to study gene function. In addition, many diseases arise from the abnormal expression of a particular gene or group of genes. Anti-mRNA agents could be used to inhibit the expression of the genes and therefore alleviate symptoms of or cure the disease. For example, genes contributing to a cancerous state could be inhibited. In addition, viral genes as well as mutant genes causing dominant genetic diseases such as myotonic dystrophy could be inhibited. Inhibiting genes such as cyclooxygenase or cytokines could also treat inflammatory diseases such as arthritis. Nervous system disorders could also be treated.

Antisense therapies hold tremendous promise for treating a wide variety of human diseases. These therapies are based on the selective inhibition of expression of specific messenger RNA or pre-messenger RNAs. Because they are highly specific, antisense agents could in theory have fewer side effects and display less toxicity than traditional drugs. In addition, because antisense agents exert their effects by binding to a complementary sequence in a target RNA molecule, designing antisense agents to specifically inhibit a particular RNA species is extremely straightforward.

A major factor hindering the effective use of antisense agents is the low efficiency at which these molecules are delivered to, and internalized by, cells in vivo. We have previously demonstrated processes that enable the delivery of plasmid DNA to the liver in vivo [Zhang et al 1999]. This technology is based on condensation of plasmid DNA by polycations resulting in plasmid DNA particles of less than 100 nm in size. An additional layer of polyanion allows “recharging” of the particle and results in increased size stability once this particle comes in contact with serum after IV injection [Trubetskoy et al. 1999a].

The delivery of genetic material has a number of useful purposes. Delivery of genes to cells both in vivo and in vitro facilitates the study of gene function. Similarly, delivery of compounds, such as antisense polynucleotides, which inhibit gene expression can also be used to study gene function. Inhibition of gene expression is useful both for basic research as well as pharmaceutical drug development.

The delivery of genetic material as a therapeutic, gene therapy, promises to be a revolutionary advance in the treatment of disease. Although, the initial motivation for gene therapy was the treatment of genetic disorders, it is becoming increasingly apparent that gene therapy will be useful for the treatment of a broad range of acquired diseases such as cancer, infectious disorders (AIDS), heart disease, arthritis, and neurodegenerative disorders (Parkinson's and Alzheimer's). Not only can functional genes be delivered to repair a genetic deficiency, but nucleic acid can also be delivered to inhibit gene expression to provide a therapeutic effect. Inhibition of gene expression can be affected by antisense polynucleotides.

A variety of methods and routes of administration have been developed to deliver pharmaceuticals that include small molecular drugs and biologically active compounds such as peptides, hormones, proteins, and enzymes to their site of action. Parenteral routes of administration include intravascular (intravenous, intra-arterial), intramuscular, intraparenchymal, intradermal, subdermal, subcutaneous, intratumor, intraperitoneal, and intralymphatic injections that use a syringe and a needle or catheter. The blood circulatory system provides systemic spread of the pharmaceutical. Polyethylene glycol and other hydrophilic polymers have provided protection of the pharmaceutical in the blood stream by preventing its interaction with blood components and to increase the circulatory time of the pharmaceutical by preventing opsonization, phagocytosis and uptake by the reticuloendothelial system. For example, the enzyme adenosine deaminase has been covalently modified with polyethylene glycol to increase the circulatory time and persistence of this enzyme in the treatment of patients with adenosine deaminase deficiency.

Transdermal routes of administration include oral, nasal, respiratory, and vaginal administration. These routes have attracted particular interest for the delivery of peptides, proteins, hormones, and cytokines, which are typically administered by parenteral routes using needles.

The ability to deliver antisense oligomers to cells has been an area of intensive research [Lebedeva et al 2000; Juliano et al 1999]. A number of reagents have been developed to increase cellular uptake and bioavailability of oligomers including cationic lipids, cationic polymers, liposomes, polypeptides, virosomes, emulsified colloids and covalently-linked sterols and porphyrins. However, many of these delivery agents are difficult to prepare or are designed for in vitro uses and are either toxic or do not translate well to in vivo delivery.

### SUMMARY

In a preferred embodiment compositions for delivery of an antisense polynucleotide to a mammalian cell in vivo are provided comprising: antisense polynucleotide, polycation and polyanion in small, negatively charged complexes that are stable in physiological salt. The complex is formed by initially associating the polynucleotide with the polycation and then recharging the complex with the polyanion. The complex is formed in a container and inserted into a vessel in the mammal. A preferred antisense polynucleotide consists of a phosphorodiamidate morpholino oligomer. A preferred polycation consists of a vinyl-amine based polycation. A preferred polyanion consists of a poly(amino acid) based polymer or a poly(maleic anhydride) polymer. A preferred cell consists of a hepatocyte.

In a preferred embodiment we describe a process for forming small serum stable particles containing antisense oligonucleotides comprising: associating the antisense polynucleotide with a polycation in a solution and adding a polyanion to the polynucleotide/polycation complex to form a small negatively charged particle. Covalent or noncovalent crosslinking of the polycation to the polyanion increases the stability of the particle. Stabilization of the particle decreases aggregation in serum, decreases aggregation at physiological salt concentration and decreases opsonization by serum components. The small, stable particle can be inserted into a vessel in a mammal for delivery to cells. A preferred cell is a liver cell. The choice of polycation and polyanion can enhance delivery of the particles to hepatocytes and decrease delivery to Kupffer cells and epithelial cells.

In a preferred embodiment we describe a process for delivery of an antisense polynucleotide to a mammalian cell in vivo comprising: forming in a solution outside the mammal a recharged small stable antisense polynucleotide-containing complex and inserting the solution into a vessel in the mammal. A preferred complex consists of a polynucleotide/polycation/polyanion complex. A preferred antisense polynucleotide consists of a phosphorodiamidate morpholino oligomer. A preferred polycation consists of a vinyl-amine based polycation. A preferred polyanion consists of a poly(amino acid) based polymer or a poly(maleic anhydride) polymer. A preferred cell consists of a hepatocyte.

In a preferred embodiment, the antisense polynucleotide-containing complex is stabilized by using a cross-linking reagent. For instance, in a ternary complex comprising a polynucleotide, a polycation, and a polyanion, the polycation may be crosslinked to itself, to the polyanion, or to the polynucleotide. The polyanion may also be crosslinked to itself, to the polycation, or to the polynucleotide.

In a preferred embodiment, the components of the complex may be modified by attachment of a functional group to enhance its extracellular or intracellular qualities. For example, endosomolytic function can be enhanced by conjugation of endosomolytic compounds. Molecules that increase cell binding or internalization or enhance cell type specific binding may also be attached to the inhibitor-containing complex. The functional group can be, but is not limited to, a targeting signal or a label or other group that facilitates delivery of the inhibitor. The group may be attached to one or more of the components prior to complex formation. Alternatively, the group(s) may be attached to the complex after formation of the complex.

In a preferred embodiment, the antisense polynucleotide may be delivered to a cell in a mammal for the purposes of inhibiting a target gene to provide a therapeutic effect. The target gene may be selected from the group comprising: dysfunctional endogenous genes. Dysfunctional endogenous genes include dominant genes which cause disease and cancer genes such as oncogenes. The antisense polynucleotide can also be delivered to a mammalian cell in vivo for the treatment of a disease or infection. The antisense polynucleotide can be delivered to reduce expression of an infectious agent gene. The inhibitor may reduce or block microbe production, virulence, or both. Delivery of the inhibitor may delay progression of disease until endogenous immune protection can be acquired. Viral genes involved in transcription, replication, virion assembly, immature viral membrane formation, extracellular enveloped virus formation, early genes, intermediate genes, late genes, and virulence genes may be targeted. Cellularly transcribed genes involved in bacterial pathogenicity may be targeted.

In a preferred embodiment, the antisense polynucleotide may be delivered to a mammalian cell in vivo to modulate immune response. Since host immune response is responsible for the toxicity of some infectious agents, reducing this response may increase the survival of an infected mammal. Also, inhibition of immune response is beneficial for a number of other therapeutic purposes, including gene therapy (where immune reaction often greatly limits transgene expression), organ transplantation, and autoimmune disorders.

In a preferred embodiment, the antisense polynucleotide may be delivered to a mammalian cell for the purpose of biological research or facilitating pharmaceutical drug discovery or target validation. Specific inhibition of a target gene can aid in determining whether inhibition of a protein or gene has a significant phenotypic effect. Specific inhibition of a target gene can also be used to study the target gene's effect on the cell.

Further objects, features, and advantages of the invention will be apparent from the following detailed description when taken in conjunction with the accompanying drawings.

### BRIEF DESCRIPTION OF THE FIGURES

FIG. 1. Illustrations of the chemical structures of: (A) poly(amino acid) based polymers, (B) vinyl-amine based polycations, (C) poly(maleic-anhydride) based polyanions, (D) poly(vinylether) based polymers, and (E) polyamide based disulfide polymer.

FIG. 2. Confocal images of liver cryosections from a mouse injected with SPLL/PLL particles containing PMO:ODN blunt-ended hybrid duplexes (left) or PMO:ODN concatamers (right). Fluorescently-labeled PMO is shown in white. Cell nuclei and peripheral actin cytoskeleton are shown in gray. Scale bar=20 μm.

FIG. 3. Cellular distribution in liver of PMO:ODN-containing particles after IV injection in mice. Fluorescently-labeled PMO is shown in white. Cell nuclei and peripheral actin cytoskeleton are shown in gray. Arrows indicate examples of PMO contained within hepatocytes. In each case, particles containing 20 μg of PMO:ODN were injected.

FIG. 4. Delivery of fluorescently labeled PMOs to livery via increased pressure tail vein injection. Fluorescently-labeled PMO is shown in white. Cell nuclei and peripheral actin cytoskeleton are shown in gray.

FIG. 5. Inhibition of CD26 expression by mCD26-1 PMO after increased pressure tail vein injection in mice. Liver sections (10 μm) are shown. Histochemical staining for CD26 activity appears dark and is localized at bile canaliculi of hepatocytes.

FIG. 6. Inhibition of firefly luciferase expression by luc-1 PMO. 2 μg of plasmid pGL3 (firefly luciferase) and 0.2 μg of plasmid pRL-SV40 (Renilla luciferase) were co-injected into mice by high pressure tail vein injection with increasing amounts of luc-1 PMO or a standard control PMO (std ctrl). One day after injection, livers were harvested and the homogenate assayed for both luciferase activities. The ratio of firefly to Renilla luciferase activities was calculated and then normalized to the ratio from mice receiving plasmids only. SD bars are shown, n=3.

### DETAILED DESCRIPTION

We have developed polymers and particle formulations for delivery of antisense polynucleotides, such as phosphorodiamidate morpholino oligomers (PMOs), to liver hepatocytes in vivo. We have found that PMO-containing recharged particles prepared with vinyl-amine polycations were of an optimal size and possessed sufficient salt stability to enable accumulation in the liver after injection into a vessel. Injection of these particles into mice led to accumulation in the liver and more importantly, internalization by hepatocytes. We show that charge density of the polycation can play an important role in determining a given particle's ability to deliver antisense polynucleotides to the liver. Antisense polynucleotide-containing particles made using low charge density polycations did not accumulate in hepatocytes.

Particle technology is premised on the creation of “artificial viruses” or “self-assembling complexes”. Preparation of particles that have virus-like characteristics should theoretically enable the efficient delivery of nucleic acids in vivo. The formation of such non-viral particles has heretofore involved simply complexing negatively charged DNA with various polycations. While viruses are discrete, non-aggregating particles, most non-viral gene transfer particles tend to aggregate in physiological solutions. The large size of these aggregates interferes with their desired biodistribution and ability to transfect cells in vivo. Excess polycation can be used to prevent aggregation, but the level of polycation required can be toxic. Even if relatively non-toxic polycations were used, it is doubtful that an excess could be maintained in vivo. Furthermore, particles with a net positive charge interact non-specifically with many blood and tissue components, preventing their contact with target cells. Probably for this reason, most viruses possess a negative ζ-potential (or surface charge).

We have previously developed processes for packaging of plasmid DNA into small particles that are stable under physiological conditions and allow for “recharging,” i.e., the formation of negatively charged particles. Recharging is accomplished by first complexing the plasmid DNA with polycations, then recharging the complex with polyanions [Trubetskoy et al. 1999a]. Particles can be stabilized by crosslinking the polymers, thus preventing aggregation in solutions containing physiological salt concentration. This technology allows the formation of particles with a negative ζ-potential thus reducing non-specific interaction with serum components in vivo. Excess polymer can be removed from the recharged particles using size exclusion chromatography or centrifugation through density gradients. Ligands, endosomal release-enhancing groups, nuclear localizing signals, and other moieties can be attached to these particles through simple chemistry.

Antisense gene inhibition strategies are premised on the use of oligonucleotides or oligonucleotide analogs designed to inhibit expression of a specific RNA. Currently, there are two broad mechanistic categories by which available antisense oligomers are believed to exert their actions: “occupancy-activated” and “occupancy-only” mechanisms [Crooke 1999]. Occupancy-activated mechanisms include RNA degradation by RNase H at the oligomer binding site, and destabilization of RNA by inhibition of 5′-capping or 3′-polyadenylation [Westermann et al 1989; Giles et al 1995]. Occupancy-only mechanisms operate through steric hindrance and include inhibition of pre-mRNA splicing and mRNA translational arrest [Dominski et al 1993; Hodges et al 1995; Kang et al 1998]. The type of mechanism by which a particular antisense oligomer exerts its effects is largely dependent on the chemical makeup of the oligomer itself. For example, phosphorothioate oligomers (S-DNAs) act primarily, but not exclusively, through occupancy-activated mechanisms dependent on RNase H activity. Phosphorodiamidate morpholino oligomers (PMOs or morpholinos) and peptide nucleic acids, on the other hand, act primarily through occupancy-only mechanisms [Giles et al 1995; Chiang et al 1991; Bonham et al 1995; Knudsen et al 1996; Giles et al 1999; Ghosh et al 2000].

Antisense PMOs have recently been shown to be extremely effective at blocking mRNA translation and preventing aberrant pre-mRNA splicing in cultured cells and in animal embryos. These antisense agents exert their effects by steric hindrance mechanisms and can be used to block translation or splicing of a target RNA [Kang et al 1998; Giles et al 1999; Ghosh et al 2000]. PMOs have an uncharged phosphorodiamidate backbone and contain nucleosides in which the ribose moiety has been converted to a six-membered morpholine ring. These modifications do not adversely affect binding/hybridization affinity for RNA. In fact, studies comparing the melting temperature of 20mer PMO:RNA hybrids with DNA:RNA or S-DNA:RNA hybrids indicate that PMO:RNA hybrids form the most stable duplexes. In comparison to other types of oligonucleotides, such as those having phosphorothioate backbones and unmodified sugars, morpholino oligonucleotides are less sensitive to nucleases, esterases and other degradative enzymes, have higher sequence specificity, and display fewer negative non-specific effects. Furthermore, PMOs exhibit better efficacy at lower concentrations likely due to the higher affinity of PMOs for RNA [Summerton et al 1997]. Finally, antisense PMOs have fairly predictable targeting characteristics [Haesman et al 2000; Matveeva et al 1998; Ho et al 1998; Milner et al 1997]. Simply targeting the PMO to a specific mRNA sequence within the first 100 base pairs 5′ to the translation start codon efficiently inhibits translation. Thus, the ability to efficiently deliver antisense PMOs targeted to specific RNA sequences in vivo would greatly expand the available research and therapeutic targets.

Because the initial step in particle formation is dependent on polycation electrostatic interaction with a negatively charged polynucleotide, neutral PMOs must be presented in a form that bears an overall negative charge. Complementary polynucleotides, such as oligodeoxynucleotides, can be utilized to form PMO:oligodeoxynucleotide hybrid duplexes which have a negative charge. The negative charge allows interactions with polycations and condensation into a particle.

For delivery in vivo, the particles must be small and stable under physiological conditions. Stability means not only the integrity of the particles, but the lack of aggregation as well. Small size facilitates exit from the vessel after intravascular administration of the particles. For example, the particles should be <200 nm in diameter in order pass though liver vessel fenestrae and gain access to and uptake by hepatocytes.

While positive surface charge may facilitate interaction between the nucleic acid/polycation complex and a cell, a negative surface charge would be more desirable for many practical applications, in particular in vivo delivery. The phenomenon of surface recharging is well known in colloid chemistry and is described in great detail for lyophobic/lyophilic systems (i.e., silver halide hydrosols). Addition of polyion to a suspension of latex particles with an oppositely-charged surface leads to the permanent absorption of the polyion onto the surface. Upon reaching appropriate stoichiometry, the surface is changed to the opposite charge.

A polynucleotide/polycation complex may be recharged by addition of a polyanion to the complex. The resulting recharged complex can be formed with an appropriate amount of charge such that the resulting complex has a net negative, positive or neutral charge. The interaction between the polycation and the polyanion can be ionic. Stabilization of the interaction can involve covalent and noncovalent crosslinking. The crosslinking can also be labile or cleavable. One of the advantages of recharging the particles is reducing their non-specific interactions with cells and serum proteins [Dash et al. 1999; Plank et al. 1996; Ogris et al. 1999; Schacht et al. 1996].

A wide a variety of polyanions can be used to recharge the polynucleotide/polycation particles comprising: succinylated PLL, succinylated PEI (branched), polyglutamic acid, polyaspartic acid, polyacrylic acid, polymethacrylic acid, polyethylacrylic acid, polypropylacrylic acid, polybutylacrylic acid, polymaleic acid, dextran sulfate, heparin, hyaluronic acid, polysulfates, polysulfonates, polyvinyl phosphoric acid, polyvinyl phosphonic acid, copolymers of polymaleic acid, polyhydroxybutyric acid, acidic polycarbohydrates, DNA, RNA, negatively charged proteins, pegylated derivatives of above polyanions, pegylated derivatives carrying specific ligands, block and graft copolymers of polyanions, any hydrophilic polymers (PEG, poly(vinylpyrrolidone), poly(acrylamide), etc), and other water-soluble polyanions

These polyanions can be added prior to the polynucleotide complex being delivered to the cell or organism. The recharged complexes can be formed in a container and administered into the vessel of an organism.

The polyanion can be covalently attached to the polycation using a variety of chemical reactions without the use of an additional crosslinker. For example, the polyanion can contain reactive groups that covalently attach to groups on the polycation.

The stability of a recharged complex, can also be enhanced by using chelators and crown ethers or by hydrophobic interactions of alkyl groups on the polymers.

The polycation can be added to the polynucleotide in excess during formation of the complex. The excess polycation that is not in complex with the antisense polynucleotide can be removed by purification of the particles prior to insertion of the particles into an organism. Similarly, the polyanion used to recharge the complex can be added in excess during particle formation. Polyanion not in association with the polynucleotide complex can be removed prior to administration in vivo. Purification means removal of charged polymer using centrifugation, dialysis, chromatography, electrophoresis, precipitation, extraction.

A function group, such as a ligand or signal, can be associated with any component of the polynucleotide/polycation/polyanion particle. The functional group can be attached to a component of the particle prior to particle formation or after particle formation.

The polymers used for particle formation can be labile or cleavable. The backbone of the polymer can be labile or attachment of side chains to the polymer backbone can be labile. A labile backbone can comprise monomers linked by labile bonds such as disulfide, diols, diazo, ester, sulfone, acetal, ketal, enol ether, enol ester, imine and enamine bonds. Attachment of side chains to the polymer can involve reactive groups (i.e. electrophiles and nucleophiles) in close proximity so that reaction between them is rapid. Examples include having carboxylic acid derivatives (acids, esters and amides) and alcohols, thiols, carboxylic acids or amines in the same molecule reacting together to make esters, thiol esters, anhydrides or amides. Examples include polymers containing an ester acid such as citraconic acid or dimethylmaleyl acid derivative that is connected to a carboxylic, alcohol, or amine group on the polyion.

Particles prepared using the methods described above should have a negative overall charge, which can be confirmed by ζ-potential measurement. Dynamic light scattering measurements can be used to determine the size of the particles. In order for particles to be of use in vivo they must be stable in physiological salt concentrations. Salt stability of the particles can be determined by measuring their size in increasing concentrations of NaCl and in physiological buffers.

A polycation is a polymer containing a net positive charge, for example poly-L-lysine hydrobromide. The polycation can contain monomer units that are charge positive, charge neutral, or charge negative, however, the net charge of the polymer must be positive. A polycation also can mean a non-polymeric molecule that contains two or more positive charges. A polyanion is a polymer containing a net negative charge, for example polyglutamic acid. The polyanion can contain monomer units that are charge negative, charge neutral, or charge positive, however, the net charge on the polymer must be negative. A polyanion can also mean a non-polymeric molecule that contains two or more negative charges.

We have synthesized amphiphilic cationic polymers that are based upon the polymerization of vinyl ethers. A vinyl ether has the general structure R₁R₂C═CR₃OR₄, where R₁, R₂, and R₃ may be any alkyl group, aryl group, or a hydrogen. R4 may be any alkyl or aryl group, but may not be hydrogen. Positive charge may be derived from the copolymerization of a phthalimide-protected amine group. Amphiphilicity is derived from the inclusion of hydrophobic vinyl ethers.

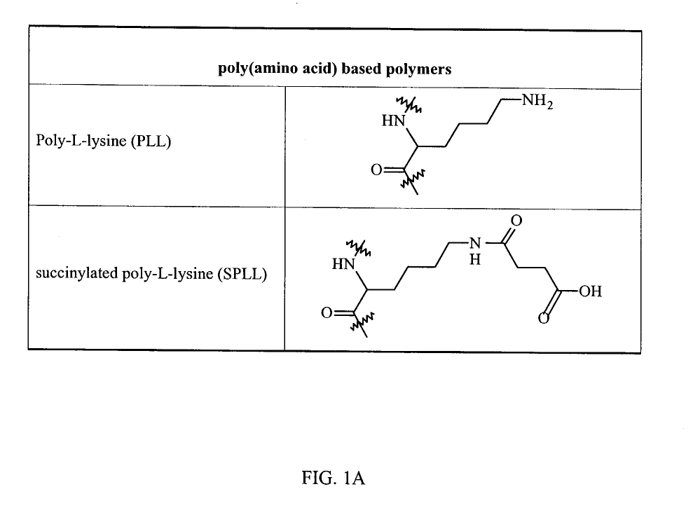

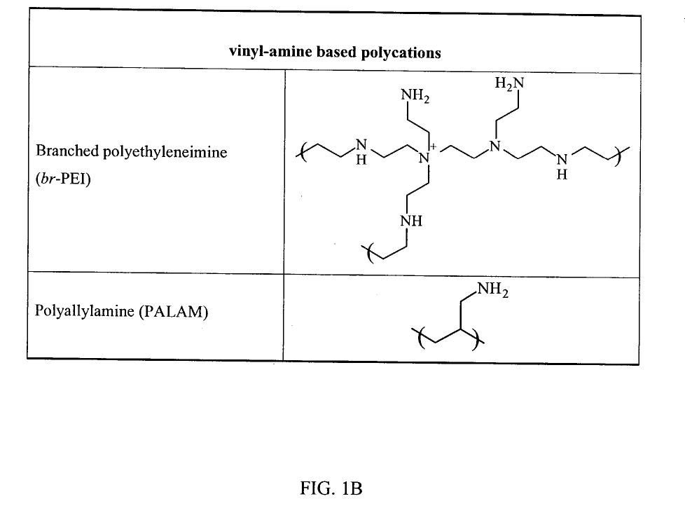

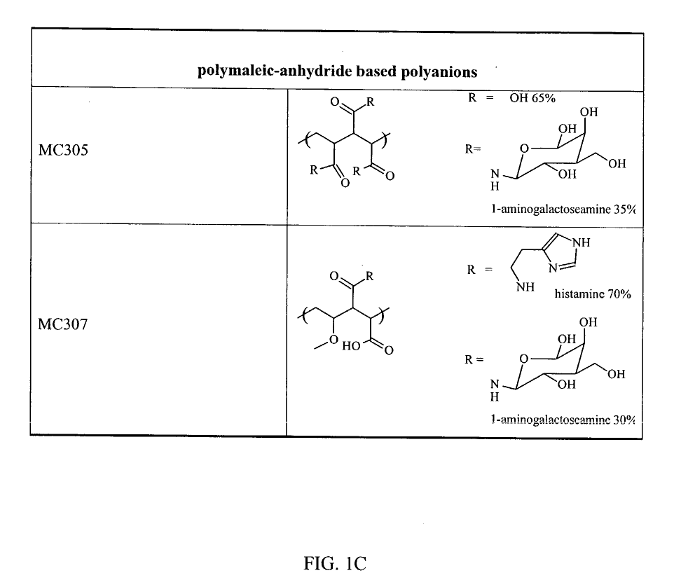

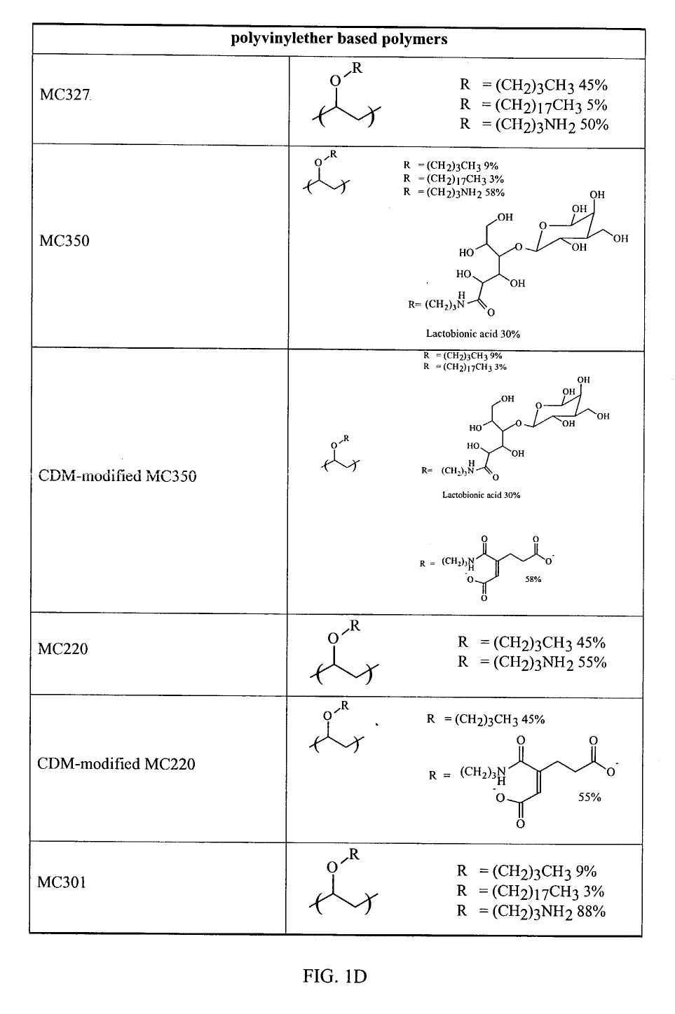

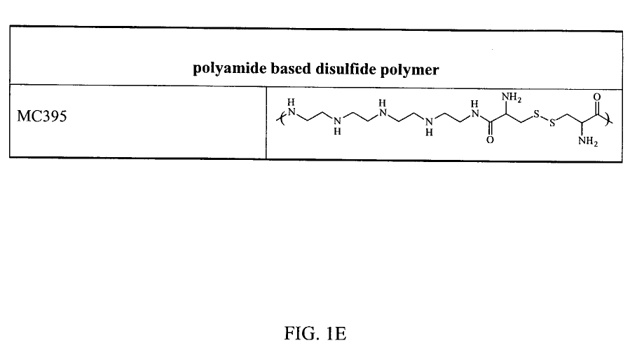

The polymerization is initiated by addition of a cationic or a Lewis acid initiator. The side-chains of the resulting polymer are a mixture of the starting vinyl ethers. In this way, polymers of varying composition are easily synthesized from mixtures of vinyl ethers. A wide variety of vinyl ethers are commercially available for example 2-chloroethyl vinyl ether, 2-aminoethyl vinyl ether, 1,4-cyclohexanedimethanol vinyl ether, 1,4-butanediol vinyl ether, 2-ethylhexyl vinyl ether, 3-amino-1-propanol vinyl ether, 4-(vinyloxy)butyl benzoate, butyl vinyl ether, cyclohexyl vinyl ether, di(ethylene glycol) vinyl ether, dodecyl vinyl ether, ethyl vinyl ether, ethylene glycol butyl vinyl ether, isobutyl vinyl ether, and octadecyl vinyl ether, which may be polymerized using this general strategy. Examples of these polymers are shown in FIG. 1D.

A variety of alkyl groups have been studied for their membrane destabilizing ability, and it has been found that alkyl groups greater than 4 carbons (butyl groups and longer) are able to disrupt membranes. Acylation of these polyvinylethers with a disubstituted maleic anhydride, 2-propionic-3-methylmaleic anhydride, generated a polyanion useful for recharging polynucleotide complexes and inhibited their membrane activity. The membrane activity may then be restored by acidification as occurs in maturing endosomes.

Synthesis of poly(maleic-anhydride) Based Polyanions.

Synthesis of Poly(Acrylic acid-co-maleic acid) Galactoseamine polymer (MC305). To solutions of Poly(Acrylic acid-co-maleic acid) (0.050 g, 0.026 mmol), and 1-amino-1-deoxy-β-D-galactose (0.1, 0.2, 0.3, 0.4 mole %) were dissolved in 5 mL 100 mM 2-[N-morpholino]-ethanesulfonic acid (MES) at pH 6.5. This solution was then added to N-(3-Dimethyl-aminopropyl)-N′-ethylcarbodiimide (EDC) (0.2, 0.3, 0.4, 0.5 mole %) followed by the addition of N-hydroxysuccinimide (NHS) (0.2, 0.3, 0.4, 0.5 mole %) in 0.5 mL 100 mM MES pH 6.5. This solution was sealed tightly and stirred for 24 h at RT. This solution was then transferred to 12,000-14,000 MWCO dialysis tubing and dialyzed against distilled water (5×5 L) over 4 days, and freeze dried. The structure of this polymer is illustrated in FIG. 1C.

Synthesis of polymaleic-anhydride based polyanion MC307 (FIG. 1C). Imidazole and galactose groups were incorporated into a polyanion by reaction of 20 mg poly(methyl vinyl ether maleic anhydride; 80,000 MW; Aldrich Chemical Co.) with 30 mg histamine and 21 mg 1-aminogalactose (7:3 mole ratio histamine to aminogalactose) in 1 mL anhydrous tetrahydrofuran. After 3 h, the solution was dissolved in 15 mL water, placed into a 12,000 MW cutoff dialysis bag, and dialyzed against 4×2 L water adjusted to pH 8 by addition of potassium carbonate for 72 h. The solution was then filtered and concentrated to a 15 mg/mL solution.

Definitions

Delivery of an antisense polynucleotide means to transfer the polynucleotide from a container outside a mammal to near or within the outer cell membrane of a muscle cell in the mammal. The term transfection is used herein, in general, as a substitute for the term delivery, or, more specifically, the transfer of a polynucleotide from directly outside a cell membrane to within the cell membrane. The antisense polynucleotide can interfere with DNA or RNA function in either the nucleus or cytoplasm.

A delivery system is the means by which a biologically active compound becomes delivered. That is all compounds, including the biologically active compound itself, that are required for delivery and all procedures required for delivery including the form (such volume and phase (solid, liquid, or gas)) and method of administration (such as but not limited to oral or subcutaneous methods of delivery).

A non-viral vector is defined as a vector that is not assembled within an eukaryotic cell including protein and polymer complexes (polyplexes), lipids and liposomes (lipoplexes), combinations of polymers and lipids (lipopolyplexes), and multilayered and recharged particles.

The term polynucleotide, or nucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations on DNA, RNA and other natural and synthetic nucleotides.

RNA function inhibitor. A RNA function inhibitor comprises any nucleic acid or nucleic acid analog containing a sequence whose presence or expression in a cell causes the degradation of or inhibits the function or translation of a specific cellular RNA, usually an mRNA, in a sequence-specific manner. Inhibition of RNA can thus effectively inhibit expression of a gene from which the RNA is transcribed. RNA function inhibitors are selected from the group comprising: siRNA, interfering RNA or RNAi, dsRNA, RNA Polymerase III transcribed DNAs encoding siRNA or antisense genes, ribozymes, and antisense nucleic acid, which may be RNA, DNA, or artificial nucleic acid. SiRNA comprises a double stranded structure typically containing 15-50 base pairs and preferably 21-25 base pairs and having a nucleotide sequence identical or nearly identical to an expressed target gene or RNA within the cell. Antisense polynucleotides include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA or linear RNAs that can function as antisense RNA. The RNA function inhibitor may be polymerized in vitro, recombinant RNA, contain chimeric sequences, or derivatives of these groups. The RNA function inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded.

Functional group. Functional groups include cell targeting signals, nuclear localization signals, compounds that enhance release of contents from endosomes or other intracellular vesicles (releasing signals), and other compounds that alter the behavior or interactions of the compound or complex to which they are attached.

Cell targeting signals are any signals that enhance the association of the biologically active compound with a cell. These signals can modify a biologically active compound such as drug or nucleic acid and can direct it to a cell location (such as tissue) or location in a cell (such as the nucleus) either in culture or in a whole organism. The signal may increase binding of the compound to the cell surface and/or its association with an intracellular compartment. By modifying the cellular or tissue location of the foreign gene, the function of the biologically active compound can be enhanced. The cell targeting signal can be, but is not limited to, a protein, peptide, lipid, steroid, sugar, carbohydrate, (non-expressing) polynucleic acid or synthetic compound. Cell targeting signals such as ligands enhance cellular binding to receptors. A variety of ligands have been used to target drugs and genes to cells and to specific cellular receptors. The ligand may seek a target within the cell membrane, on the cell membrane or near a cell. Binding of ligands to receptors typically initiates endocytosis. Ligands include agents that target to the asialoglycoprotein receptor by using asialoglycoprotein or galactose residues. Other proteins such as insulin, EGF, or transferrin can be used for targeting. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as lipids, fatty acids, cholesterol, dansyl compounds, and amphotericin derivatives. In addition viral proteins could be used to bind cells.

After interaction of a compound or complex with the cell, other targeting groups can be used to increase the delivery of the biologically active compound to certain parts of the cell.

Nuclear localizing signals enhance the targeting of the pharmaceutical into proximity of the nucleus and/or its entry into the nucleus during interphase of the cell cycle. Such nuclear transport signals can be a protein or a peptide such as the SV40 large T antigen NLS or the nucleoplasmin NLS. These nuclear localizing signals interact with a variety of nuclear transport factors such as the NLS receptor (karyopherin alpha) which then interacts with karyopherin beta. The nuclear transport proteins themselves could also function as NLS's since they are targeted to the nuclear pore and nucleus. For example, karyopherin beta itself could target the DNA to the nuclear pore complex. Several peptides have been derived from the SV40 T antigen. Other NLS peptides have been derived from the hnRNP A1 protein, nucleoplasmin, c-myc, etc.

Many biologically active compounds, in particular large and/or charged compounds, are incapable of crossing biological membranes. In order for these compounds to enter cells, the cells must either take them up by endocytosis, i.e., into endosomes, or there must be a disruption of the cellular membrane to allow the compound to cross. In the case of endosomal entry, the endosomal membrane must be disrupted to allow for movement out of the endosome and into the cytoplasm. Either entry pathway into the cell requires a disruption of the cellular membrane. Compounds that disrupt membranes or promote membrane fusion are called membrane active compounds. These membrane active compounds, or releasing signals, enhance release of endocytosed material from intracellular compartments such as endosomes (early and late), lysosomes, phagosomes, vesicle, endoplasmic reticulum, Golgi apparatus, trans golgi network (TGN), and sarcoplasmic reticulum. Release includes movement out of an intracellular compartment into the cytoplasm or into an organelle such as the nucleus. Releasing signals include viral components such as influenza virus hemagglutinin subunit ITA-2 peptides and other types of amphipathic peptides. The control of when and where the membrane active compound is active is crucial to effective transport. If the membrane active agent is operative in a certain time and place it would facilitate the transport of the biologically active compound across the biological membrane. If the membrane active compound is too active or active at the wrong time, then no transport occurs or transport is associated with cell rupture and cell death. Nature has evolved various strategies to allow for membrane transport of biologically active compounds including membrane fusion and the use of membrane active compounds whose activity is modulated such that activity assists transport without toxicity. Many lipid-based transport formulations rely on membrane fusion and some membrane active peptides' activities are modulated by pH. In particular, viral coat proteins are often pH-sensitive, inactive at neutral or basic pH and active under the acidic conditions found in the endosome.

Another functional group comprises compounds, such as polyethylene glycol, that decrease interactions between molecules and themselves and with other molecules. Such groups are useful in limiting interactions such as between serum factors and the molecule or complex to be delivered.

### EXAMPLES

1. Demonstration of formation of recharged particles containing PMOs. In order to form PLL/SPLL particles with PMOs, we annealed a fluorescein-labeled PMO 25mer (GeneTools Corp.) with oligodeoxynucleotides (ODNs) of complementary sequence in order to form negatively charged PMO:ODN hybrid duplexes. We prepared two different types of these hybrid duplexes to be used in particle formation: 25-mer hybrid duplexes with blunt ends and longer concatenated hybrid duplexes. To form concatamers, we annealed the PMO to an ODN with sequence in its 5′ and 3′ halves designed to be complementary to the 5′ and 3′ ends of the PMO, respectively. Using a 25mer PMO, the ODN was designed to base pair with 13 bases at the 3′ end of the first morpholino oligonucleotide and 12 bases of the 5′ end of the next morpholino oligonucleotide. Alternatively, to increase the effective length of the morpholino, it is possible to anneal many PMOs on a complementary DNA consisting of many repeats of the sequence complementary to the PMO. Hybridization of the PMO to the ODN designed to create blunt-ended duplexes or to the ODN designed to create the concatamers was confirmed by gel electrophoresis. Results indicated that after hybridization with equimolar amounts of PMO and ODN, there was no detectable free PMO. An estimate of the size of the concatamers ranged between that expected for the monomer to that expected for concatamers composed of 10 PMO:ODN monomers.

Each of the hybrid duplexes was complexed with PLL at a charge ratio of 1:3. To recharge the particles, EDC:sulfo-NHS (Pierce) pre-activated S-PLL polyanion was complexed with the (PMO:ODN)/PLL complexes at a charge ratio of 1:3:10 (PMO:ODN/PLL/SPLL). Activation of the S-PLL polyanion allows covalent crosslinking of the polyanion with the amines present on the polycations. Crosslinking was allowed to proceed for 2 h at RT. The crosslinked particles were purified on a Sephadex G-25 spin column to remove unreacted molecules. Recharged particles containing either blunt-ended duplexes or concatamers could be formed. Dynamic light scattering measurements revealed that the average size of the particles prepared with the blunt-ended hybrid duplexes was 101 nm. The size of particles prepared with the concatamers was 225 nm.

2. Delivery of recharged PMO:ODN/PLL/SPLL particles to liver following intravascular administration. A key factor which will determine whether or antisense-polynucleotide particles will be useful as in vivo delivery vehicles is their ability to accumulate in a target tissue. Crosslinked SPLL/PLL particles containing 30 μg of the blunt-ended or concatenated PMO:ODN hybrid duplexes in 250 μl were injected into the tail vein of mice. In these experiments, the PMO was labeled with fluorescein at the 3′ end. Labeling the PMO did not significantly affect particle formation. After 24 h, the mice were sacrificed and the livers were harvested. Examination of liver cryosections by confocal microscopy revealed the presence of fluorescein-labeled PMO within endothelial and Kupffer cells (FIG. 2). No labeled PMOs could be observed in the liver when injected alone or as PMO:ODN duplexes. The staining pattern was punctate, consistent with that observed for particles containing plasmid DNA. These results indicate particles containing PMO:ODN hybrid duplexes have sufficient stability to be delivered to the liver in vivo. However, unlike particles containing plasmid DNA, particles containing PMOs did not deliver detectable amounts of PMOs to hepatocytes.

Liver sectioning, fixing and confocal microscopy: Frozen liver sections, 4-5 μm thick, were prepared and fixed for 15 minutes in PBS containing 3.6% formaldehyde at RT. The tissue sections were then washed 3×5 minutes in PBS. Nuclei were counterstained with ToPro-3 (Molecular Probes) and cytoskeletal actin counterstained with Alexa 568 phalloidin (Molecular Probes) for 20 min in PBS at RT. The sections were washed 1×5 minutes in PBS and mounted in VectaShield (Vector Laboratories).

3. Development of antisense oligonucleotide-containing particles that target hepatocytes following intravascular administration. We have found that if a polymer does not properly associate with the charge dense, high molecular weight plasmid DNA, then that polymer will behave even less well when complexed with low molecular weight and less charge dense PMO:ODN hybrid oligonucleotides. However, polymers that form small stable complexes with plasmid DNA do not necessarily form small stable particles with oligonucleotides.

A number of polymers were synthesized and tested for delivery PMO:ODN duplexes to cells. The chemical structures of these polymers are shown in FIG. 1. PMO:ODN-containing particles were formed using these polymers and the particles were characterized with respect to size, salt stability, and transfection ability. Particles were prepared with PMO hybridized to ODN to form concatemers (DL10), blunt-ended duplexes (DL28), or duplexes in which the ODNs were complementary to the 5 ′ 16 bases of the PMO (DL47). Particles were also recharged with different polyanions. Particles prepared with a given polymer did not appear to significantly differ with respect to size in low or high salt for the different PMO:ODN hybrid configurations. The length of the ODN or the particular base pairing scheme (i.e. concatemer (DL10), blunt-ended hybrid duplexes (DL28) or partially hybridized morpholino (DL47)) had little effect on size or stability of particles prepared with these polymers. The sizes of particles in low (0 mM) salt are sufficiently small to enable liver targeting in vivo (Table 1). Furthermore, the particles do not aggregate or increase in size when exposed to physiological (150 mM) salt concentrations.

TABLE 1
Size of recharged particles, in nm, prepared
on PMO:ODN hybrid duplexes.

| | | Polycation/Polyanion | | | | |
|---|---|---|---|---|---|---|
| | NaCl | PLL/ | br-PEI/ | MC395/ | PALAM/ | PALAM/ |
| PMO:ODN | (mM) | S-PLL | S-PLL | S-PLL | MC305 | MC307 |
| DL10 | 0 | 102.7 | 92.7 | 151.3 | 106.6 | 119.3 |
| | 150 | 100.3 | 90.1 | 189.3 | 106.5 | 122.4 |
| DL28 | 0 | 107.0 | 119.5 | 254.2 | 109.0 | 113.7 |
| | 150 | 102.3 | 119.5 | ND | 108.4 | 118.6 |
| DL47 | 0 | 85.2 | 113.2 | 237.8 | 111.6 | 113.3 |
| | 150 | 87.9 | 108.3 | ND | 113.6 | 111.5 |

In contrast, particles prepared with a disulfide-containing polymer, MC395 did show differences in size and salt stability depending on the PMO:ODN configuration. The smallest and most salt stable particles were those prepared with the PMO:ODN concatemer (DL10), suggesting the larger theoretical size of this PMO:ODN hybrid may impart greater stability to the particle. These results indicate that particles prepared with the indicated polycations are of an acceptable size (<150 nm) and are of sufficient salt stability to be good candidates for delivery to the liver.

ζ-potential and size measurements are performed in a Zeta Plus Particle Analyzer (Brookhaven Instruments Corp., Holtsville, N.Y.), with a laser wavelength of 532 nm. The size, charge and the extent of aggregation of the particles in buffer containing increasing NaCl concentrations, and a physiological buffer (“intracellular” buffer: 10 mM PIPES, 140 mM KCl, 1 mM MgCl2) is determined using dynamic light scattering.

Next, the particles were tested for their ability to delivery antisense oligonucleotides to hepatocytes in vivo. Particles displaying satisfactory size and sufficient stability in physiological salt concentrations as well as polymers of other classes were tested. Because no significant difference in the physical characteristics between particles prepared with the three different PMO:ODN configurations, all subsequent particle formulations were prepared using the PMO:ODN concatemer. This hybrid configuration gave the highest antisense activity in in vitro assays (data not shown).

Four different classes of polymers were utilized, polyamino based, vinyl-amine based, polymaleic-anhydride based and polyvinylether based. These classes were chosen because of their charge density, their ability to allow attachment of amine-containing molecules for liver targeting (e.g. 1-aminogalactoseamine) or allow crosslinking of polyion layers. In some cases, the polycation and polyanion were cross-linked with EDC (N-(3-Dimethylamino-propyl)-N′-ethylcarbodiimide Hydrochloride) in order to add stability to the molecule during transit through the bloodstream. In one case (br-PEI/MC307(10₄)), crosslinking was accomplished using periodate. Sodium periodate oxidizes vicinal hydroxyl groups, such as those found on carbohydrates, to form two aldehyde groups, which may then form Schiff bases with amine groups. In this way, carbohydrate-containing polyanions may be linked to amine-containing polycations. Crosslinking is not necessary with the polyvinylether polymers, which are stabilized by hydrophobic interactions. Polyvinylethers can also be modified with maleic anhydride derivatives in order to impart a negative charge and to add lability to the polyanion. Dimethylmaleic anhydride reacts with amines to form acid-labile dimethylmaleamate groups, which are much more labile than the corresponding monosubstituted maleamates formed from citraconic anhydride (methylmaleic anhydride). In order to make dimethylmaleamates more water soluble, we synthesized 2-propionic-3-methylmaleic anhydride (carboxy dimethylmaleic anhydride or CDM). The physical characteristics of particles prepared for in vivo injection are shown in Table 2. In all cases, particles prepared in low salt conditions are of a size expected to allow access to the liver via the vasculature. Most particle formulations are also salt stable with size increasing only slightly in the presence of 150 mM NaCl.

TABLE 2
Size and stability of recharged PMO-ODN-containing particles
made with various polycation and polyanions.

| | | | | Particle size (nM) | |
|---|---|---|---|---|---|
| Formulation | +UZ,8/210polymer class | | cross- | 0 mM | 150 mM |
| (X-link) | polycation | polyanion | linker | NaCl | NaCl ^{a} |
| PLL/S-PLL | polyamino | | EDC | 102.7 | 100.3 |
| br-PEI/S-PLL | vinyl-amine | polyamino | EDC | 92.7 | 90.1 |
| PALAM/305 | ″ | ″ | EDC | 106.6 | 106.5 |
| PALAM/307 | ″ | ″ | EDC | 119.3 | 122.4 |
| 327/CDM-350 | polyvinylether | CDM- | | 120.0 | 122.6 |
| | | polyviny- | | | |
| | | lether | | | |
| 301/CDM-350 | polyvinylether | CDM- | | 110.7 | 138.2 |
| | | polyviny- | | | |
| | | lether | | | |
| 220/CDM-220 | polyvinylether | CDM- | | 149.3 | 157.6 |
| | | polyviny- | | | |
| | | lether | | | |

^{a} after 10 min at RT

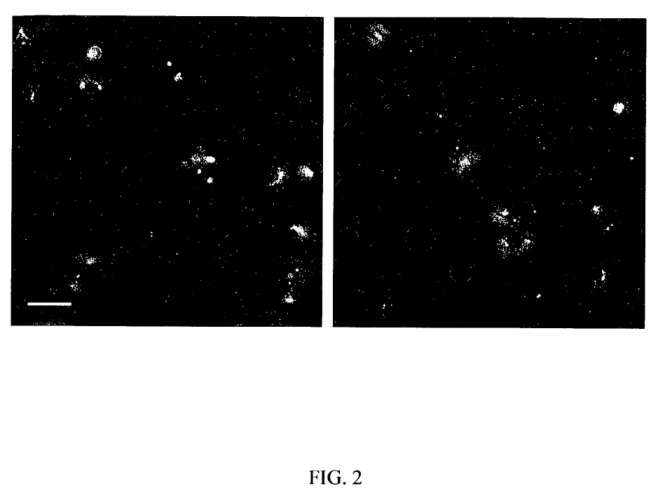

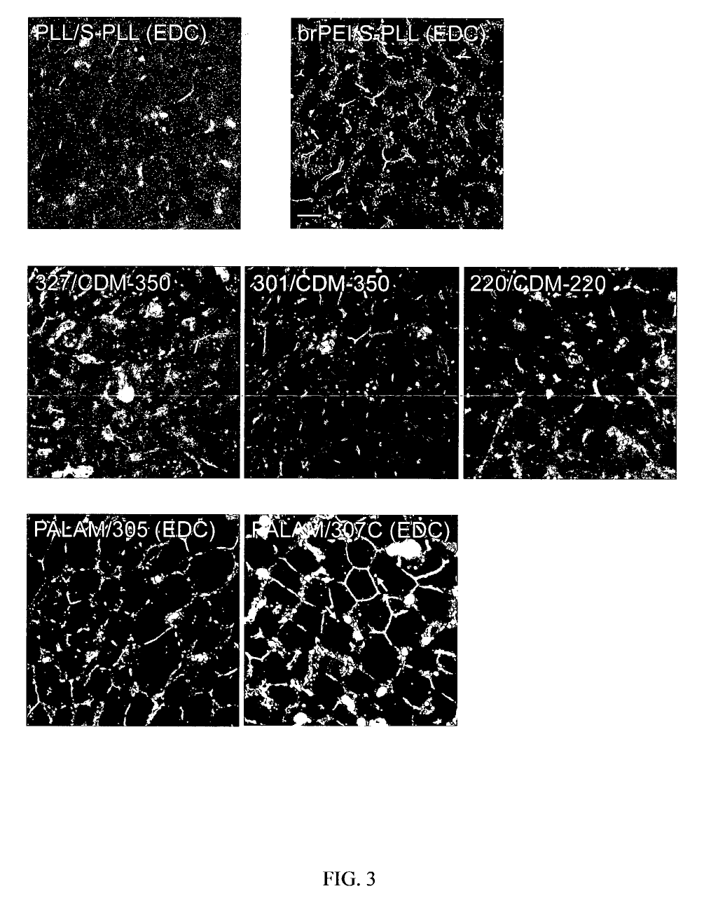

These particles were then tested for delivery of PMO:ODN duplexes to hepatocytes in vivo by injection into the tail vein of mice. Three hours after injection, the mice were sacrificed and the livers harvested for sectioning. The PMO was labeled with FITC for visualization in liver sections by confocal microscopy. A representative subset of the results of these experiments is shown in FIG. 3.

The distribution of the PLL/S-PLL (EDC) particles is shown as a reference. As indicated above, these particles primarily localized to Kupffer cells, with little to no hepatocyte delivery, Similarly, polyvinylether polymer-based particles also accumulated primarily in Kupffer cells and not in hepatocytes. In contrast, recharged particles prepared with the vinyl amine polycations successfully targeted hepatocytes in vivo. One explanation for these observations is that the vinyl amines-based polycations (PALAM, br-PEI) are more charge dense than either the polyamino or polyvinylether polycations. A greater charge density would allow for a more stable interaction with the PMO:ODN.

The most effective particle for PMO:ODN delivery to hepatocytes is the recharged crosslinked br-PEI/S-PLL(EDC) formulation. This formulation results in the greatest number of hepatocytes containing PMO:ODN with minimal amounts of particles in Kupffer cells. Heavy accumulation of particles in Kupffer cells may lead to liver toxicity. If the polycation PALAM is used, fewer particles reach hepatocytes and large amounts of PMO:ODN are observed in Kupffer cells. Hepatocyte uptake of PALAM-containing particles was dependent on the nature of the polyanion used to recharge the particles. Hepatocyte uptake is observed if the polymaleic-anhydride anionic polymer MC307 is used but not when the polymaleic-anhydride anionic polymer MC305 is used. MC307 differs from MC305 in the presence of histamine in MC307 in place of a hydroxyl in MC305. This histamine may assist in targeting the particle to hepatocytes or assist in hepatocyte uptake of the particle.

4. Intravascular delivery of naked PMOs to hepatocytes by increased pressure tail vein injection. Injection of naked plasmid DNA into the tail vein of mice has been shown to be an efficient method for hepatocyte transfection in vivo [Zhang et al. 1999; Liu et al. 1999]. We tested this method for the ability to deliver PMOs to the liver. Briefly, 30 μg of fluorescein labeled PMO or PMO:ODN hybrid duplexes in 1-2.5 ml Ringer's solution (147 mM NaCl, 4 mM KCl, 1.13 mM CaCl₂) were injected into the tail vein of mice in 4-5 sec using a 27 gauge needle. 24 h after injection, the animals were sacrificed and the livers harvested. Tissue was immediately placed in embedding compound and snap frozen in liquid nitrogen. Cryosections (10 μm) were fixed for 15 min in 3.6% formaldehyde in PBS, pH 7.4, rinsed 3×5 minutes in PBS and then counterstained for 20 minutes with a 1:40,000 dilution of the nucleic acid stain, ToPro-3 (Molecular Probes). After rinsing with PBS, the sections were mounted in VectaShield (Vector Laboratories, Inc.) and examined using a Zeiss LSM 510 confocal microscope.

Using this technique, approximately 10% of hepatocytes were observed to contain PMO (FIG. 4). In contrast, we observed no PMO uptake by liver cells following tail vein injection using 250 μl volume (data not shown). No difference in the extent of PMO uptake was observed when the PMO was delivered alone or in PMO:DNA hybrid duplexes. The intracellular distribution was diffuse cytoplasmic accompanied by accumulation in the nucleus. These results indicate that tail vein injections can be used to deliver PMOs to hepatocytes when a sufficient volume is injected at a sufficient rate. More importantly, using this method, PMOs are delivered to the cytoplasmic and nuclear compartments, the cellular locations of PMO action.

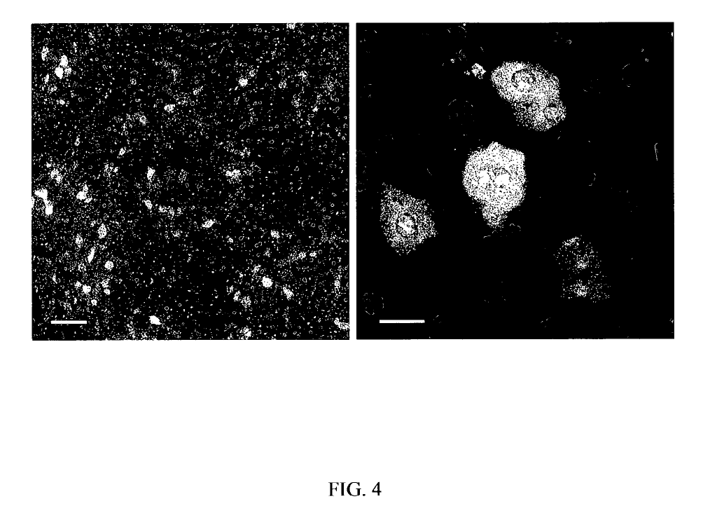

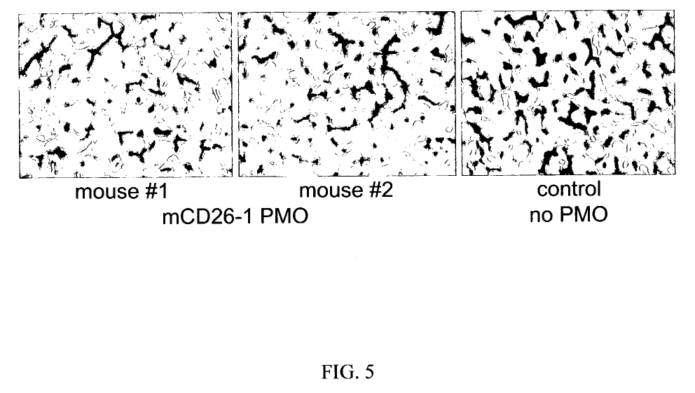

5. Inhibition of gene expression in liver hepatocytes following intravascular delivery of PMOs. The PMO, mCD26-1, was designed to base pair to positions −3 to +20 of the mouse CD26 coding sequence. Mice were injected into the tail vein as above with a 2.5 ml solution containing either 100 μg mCD26-1 or no oligonucleotide. Using histochemical staining, an overall decrease in CD26 activity could be observed in animals receiving the mCD26-1 antisense PMO compared (FIG. 5). Some areas of each section appeared to contain higher levels of CD26 gene expression inhibition than others and may reflect the uneven distribution of the PMOs after injection using this method.

Animals were sacrificed and their livers harvested. Tissues were immediately placed in embedding compound and snap frozen in liquid nitrogen. Cryosections (10 μm) were fixed for 15 min in 3.6% formaldehyde in PBS, pH 7.4 at RT and then rinsed 3×5 minutes in PBS. The fixed cryosections will be placed in blocking buffer (PBS, 1% BSA) for 1 h at RT. The cryosections were be incubated in a 1:1000 dilution of the goat anti-CD26 primary antibody (Research Diagnostics) in blocking buffer at 40° C. overnight. After 3×20 min washes in blocking buffer, the cryosections were incubated in a 1:400 dilution of Cy-3 conjugated donkey anti-goat F(ab′)2 fragments (Jackson Laboratories) and 1:40,000 dilution of the nuclear stain ToPro-3 (Molecular Probes) in blocking buffer for 2 h at RT. After rinsing with PBS, the sections were mounted in VectaShield (Vector Laboratories, Inc.) and examined using a Zeiss LSM 510 confocal microscope.

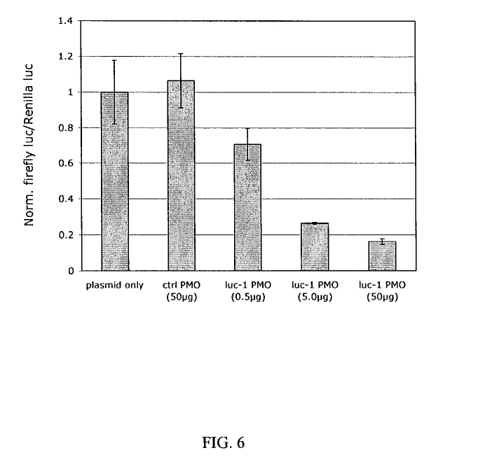

Similarly, intravascular tail vein injection was used to co-deliver a firefly luciferase expression plasmid (pGL3) and an expression plasmid containing the unrelated Renilla luciferase (pRL-SV40) along with a PMO designed to base pair to positions −3 to +22 of the firefly luciferase coding region (Luc-1 PMO), a control PMO or no PMO. The Renilla luciferase served as an internal control to normalize for plasmid delivery efficiency. 24 h after injection, the activities of both luciferase enzymes in liver homogenates were assayed. The ratio of firefly luciferase activity to Renilla luciferase activity was then compared with the ration obtained in mice injected with solution containing plasmids only. Results are shown in FIG. 6. In mice are co-injected with increasing amounts of luc-1 PMO, increasing amounts of firefly luciferase expression inhibition was observed. The greatest inhibition, 83%, was observed in mice receiving 50 μg luc-1 PMO. No inhibition was observed in mice receiving the control PMO.

We have thus demonstrated gene-specific inhibition of expression following intravascular delivery of PMOs using the describe technique.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

### REFERENCES

1. Bonham M A, Brown S, Boyd A L, Brown P H, Bruckenstein D A, Hanvey J C, Thomson S A, Pipe A, Hassman F. Nucleic Acids Res. 23:1197-1203, 1995.

2. Boussif O, Lezoualc'h F, Zanta M A, Mergny M D, Scherman D, Demeneix B, Behr J P. Proc Natl Acad Sci USA. 92:7297-7301, 1995.

3. Chiang M Y, Chan H, Zounes M A, Freier S M, Lima W F, Bennett C F. J Biol Chem, 266:18162-18171, 1991.

4. Crooke S T. Biochim Biophys Acta 1489:31-44, 1999.

5. Dash P R, Read M L, Barrett L B, Wolfert M A, Seymour L W. Factors affecting blood clearance and in vivo distribution of polyelectrolyte complexes for gene delivery. Gene Ther. 6(4):643-50, 1999.

6. Dominski Z, Kole R. Proc Natl Acad Sci USA 90:8673-8677, 1993.

7. Escriou V, Ciolina C, G. Byk G, Scherman D, Wils P. Biochim Biophys Acta. 1368:276-288, 1998.

8. Ghosh C, Stein D, Weller D, Iversen P. Methods Enzymol 313:135-143, 2000.

9. Giles R V, Spiller D G, Clark R E, D.M. Tidd D M. Antisense Nucleic Acid Drug Dev 9:213-220, 1999.

10. Giles R V, Spiller D G, Tidd D M. Antisense Res Dev 5:23-31, 1995.

11. Heasman J, Kofron M, Wylie C. Dev Biol 222:124-134, 2000.

12. Ho S P, Bao Y, Lesher T, Malhotra R, Ma L Y, Fluharty S J, Sakai R R. Nat Biotechnol 16:59-63, 1998.

13. Hodges D, Crooke S T. Mol Pharmacol 48:905-918, 1995.

14. Juliano R L, Alahari S, Yoo H, Kole R, Cho M. Pharm Res 16:494-502, 1999.

15. Kang S H, Cho M J, Kole R. Biochemistry 37:6235-6239, 1998.

16. Knudsen H, Nielsen P E. Nucleic Acids Res 24:494-500, 1996.

17. Lebedeva I, Benimetskaya L, Stein Calif., Vilenchik M. Eur J Pharm Biopharm 50: 101-119, 2000.

18. Liu F, Song Y, Liu D. Gene Ther. 6:1258-1266, 1999.

19. Matveeva O, Felden B, Tsodikov A, Johnston J, Monia B P, Atkins J F, Gesteland R F, Freier S M. Nat Biotechnol 16:1374-1375, 1998.

20. Milner N, Mir K U, Southern E M. Nat Biotechnol 15:537-541, 1997.

21. Ogris M, Brunner S, Schuller S, Kircheis R, Wagner E. PEGylated DNA/transferrin-PEI complexes: reduced interaction with blood components, extended circulation in blood and potential for systemic gene delivery. Gene Ther. 6(4):595-605, 1999.

22. Plank C, Mechtler K, Szoka F C Jr, Wagner E. Activation of the complement system by synthetic DNA complexes: a potential barrier for intravenous gene delivery. Hum Gene Ther. 7(12):1437-46, 1996.

23. Schacht et al. Brit. Patent Application 9623051.1, 1996

24. Summerton J, Stein D, Huang S B, Matthews P, Weller D, Partridge M. Antisense Nucleic Acid Drug Dev 7:63-70, 1997.

25. Trubetskoy V S, Loomis A, Hagstrom J E, Budker V G, Wolff J A. Layer-by-layer deposition of oppositely charged polyelectrolytes on the surface of condensed DNA particles. Nucleic Acids Research. 27(1):3090-3095, 1999a.

26. Trubetskoy V S, Loomis A, Slattum P M, Hagstrom J E. Caged DNA Does Not Aggregate in High Ionic Strength Solutions. Bioconjugate Chem. 10:624-628, 1999b.

27. Westermann P, Gross B, Hoinkis G. Biomed Biochim Acta 48:85-93, 1989.

28. Xu Y, Szoka F C Jr. Mechanism of DNA release from cationic liposome/DNA complexes used in cell transfection. Biochemistry. 35(18):5616-5623, 1996.

29. Zhang G, Budker V, Wolff J A. High Levels of Foreign Gene Expression in Hepatocytes after Tail Vein Injections of Naked Plasmid DNA. Human Gene Therapy. 10:1735-1737, 1999.

We claim:

1. A composition for delivering an antisense oligonucleotide to a cell in vivo comprising:
an antisense oligonucleotide, a polycation, and a polyanion wherein the oligonucleotide, polycation and polyanion associate to from ζ-negative complex.

2. The composition of claim 1 wherein the polycation consists of a vinyl-amine based polymer.

3. The composition of claim 2 wherein the vinyl-amine based polymer consists of polyethyleneimine.

4. The composition of claim 3 wherein the polyethyleneimine consists of branched polyethyleneimine.

5. The composition of claim 2 wherein the vinyl-amine based polymer consists of polyallylamine.

6. The process of claim 1 wherein the polyanion consists of a poly(amino acid) polymer.

7. The process of claim 6 wherein the poly(amino acid) based polymer consists of succinylated poly-L-lysine.

8. The process of claim 1 wherein the polyanion consists of a poly(maleic anhydride) based polyanion.

9. The composition of claim 8 wherein the polyanion consists of a histamine containing poly(maleic anhydride) based polyanion.

10. The composition of claim 9 wherein the histamine containing poly(maleic anhydride) based polyanion consists of MC307.

11. The composition of claim 1 wherein the antisense polynucleotide consists of a phosphorodiamidate morpholino oligomer.

12. The composition of claim 11 wherein the phosphorodiamidate morpholino oligomer consists of a phosphorodiamidate morpholino oligomer hybridized to a oligodeoxynucleotide

13. The composition of claim 1 wherein the antisense polynucleotide consists of a peptide nucleic acid.

14. The composition of claim 1 wherein the cell consists of a mammalian liver cell.

15. The process of claim 14 wherein the liver cell consists of a hepatocyte.
